(19) United States
(12) Patent Application Publication
(10) Pub. No.: US 2024/0238112 A1
(43) Pub. Date: Jul. 18, 2024

(54) INTEGRALLY FORMED DEVICE FOR MITIGATION OF TEMPOROMANDIBULAR JOINT DISORDER

(71) Applicant: TMJ Services, LLC, Brentwood, TN (US)

(72) Inventor: Anthony Philip Urbanek, Franklin, TN (US)

(21) Appl. No.: 18/414,013

(22) Filed: Jan. 16, 2024

Related U.S. Application Data
(60) Provisional application No. 63/439,240, filed on Jan. 16, 2023.

(51) Int. Cl. A61F 5/058; A61F 5/56

(52) U.S. Cl. CPC A61F 5/05891; A61F 2005/563

(57) ABSTRACT

A device for mitigation of temporomandibular joint disorder includes a middle portion configured for placement in an upper jaw of a subject, at least one tooth receptacle extending from the middle portion and shaped to receive a corresponding upper posterior tooth, a top surface of the middle portion configured to communicate with a hard palate of the subject and lingual surfaces of four or more anterior teeth of the upper jaw, and a bottom surface of the middle portion having an anterior pad configured such that voluntary forward translational movement of the lower jaw teeth relative to the middle portion can be freely accomplished.

### CROSS-REFERENCE TO RELATED APPLICATIONS

This application claims priority to U.S. Provisional Patent Application No. 63/439,240 filed Jan. 16, 2023, the contents of which are hereby incorporated by reference in their entirety.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### BACKGROUND OF THE INVENTION

This invention relates generally to the field of dental appliances and more specifically to a device for mitigation of temporomandibular joint disorder.

The temporomandibular joint is the joint between the mandible, or lower jaw, and the skull, particularly at a portion of the temporal bone.

The joint allows rotary motion of the jaw and translational motion, side to side, of the mandible, which allows a variety of functions involving lower jaw movement. As are all joints, the temporomandibular joint is susceptible to dysfunctions of various sorts.

For example, such common anomalies as bruxism, (repetitive unconscious clenching or grinding of teeth, often during sleep), malalignment of the occlusal surfaces of the teeth, jaw thrusting, degenerative joint disease, or insufficient overbite can cause excess stress on the temporomandibular joint. Said stress may cause damage to joint elements causing undesirable effects such as biting or chewing difficulty; discomfort; clicking, popping, or grating sounds accompanying jaw movement; dull, aching pain in the face; earaches; headaches including migraines; hearing loss; jaw pain; a reduced ability to open and close the mouth; tinnitus; and neck and/or shoulder pain. Said symptoms may be referred to generically as temporomandibular joint disorder.

Remedies for temporomandibular joint disorder include analgesic drugs of various sorts, manual adjustment of the teeth by grinding, reconstructive dentistry, orthodontics, arthrocentesis, surgical repositioning of jaws, replacement of the jaw joints with implants, muscle relaxation therapy, and hypnotherapy, among others. While such therapies can yield satisfactory results, they can be expensive, radically invasive, painful and otherwise uncomfortable. They may require extended periods of time during which no relief of symptoms is realized. Thus, they are generally considered undesirable, especially if relatively inexpensive and non-invasive alternatives are available. Occlusal splints, or dental appliances, comprise such alternatives. The instant art is a novel and counterintuitive advancement in the art thereof.

Occlusal splints for the mitigation of temporomandibular joint disorder are known and in use. For example, the following U.S. patents describe a number of variations of such occlusal splints: U.S. Pat. No. 7,607,438 B2 by Pelerin, U.S. Pat. No. 4,773,853 by Kussick, U.S. Pat. No. 5,511,562 by Hancock, U.S. Pat. No. 5,203,701 by Burtch, U.S. Pat. No. 4,568,280 by Ahlin, U.S. Pat. No. 4,810,192 by Williams, U.S. Pat. No. 5,173,048 by Summer, U.S. Pat. No. 5,066,226 by Summer, U.S. Pat. No. 6,978,786 B2 by Sabbagh, U.S. Pat. No. 5,368,477 by Neely, U.S. Pat. No. 4,211,008 by Lerman, U.S. Pat. No. 6,237,601 B1 by Kittelson et al., U.S. Pat. No. 5,879,155 by Kittelson, U.S. Pat. No. 5,584,687 by Sullivan et al., U.S. Pat. No. 7,234,467 B2 by Ball, U.S. Pat. No. 7,730,891 B2 by Lamberg, and Japan Abstract JP 2009082670 (A) by Masuhiro.

The above prior art teach devices that engage facial surfaces of the anterior upper teeth, involve multi-layer construction, and/or include moldable material, elements engaging occlusal surfaces of lower posterior teeth, downwardly extending projections, upper and lower compartments, a lower member engageable with an upper member, an web connecting channels that receive teeth, a mouth guard, extensible and retractable elements, fluid-filled pads and tubes for fluid transfer between pads, disposable cushions, a groove to receive teeth of the opposing dental arch, an element having contour conforming to condylar motion, and/or a protrusive element depending from the main body of the device. The devices taught by the prior art interfere with speech or breathing and/or are complex, heavy, expensive, uncomfortable to wear, and/or clearly visible when worn.

U.S. Pat. No. 9,314,320 B2 by Urbanek discloses another variation of an occlusal splint that was an improvement over the above prior art. However, Urbanek '320 discloses ball clasps and friction for securing the device in the mouth. Securing the device through friction requires precise conformity between the device and all surfaces of the mouth such that there is sufficient contact between the device and the mouth to retain the device in an operative position. However, slippage is still possible and common when device is secured solely by friction, and the device may have to be repeatedly repositioned in the mouth into an operative position. Securing the device in the mouth via ball clasps generally requires a two-step manufacturing process whereby the majority of the device may be formed through one process and the ball clasps must be formed through a separate process and later attached to the rest of the device. When ball clasps are utilized, the segments of the clasps that extend between the premolars tend to create a pivot point, or fulcrum axis, about which the device can rock slightly when worn. Further, metal ball clasps can tend to weaken and lose retention over time from repeated flexing when inserting and removing.

What is needed then are improvements to devices for mitigation of temporomandibular joint disorder.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The primary object of the invention is to provide a temporomandibular joint disorder alleviation device that is comfortable to wear.

Another object of the invention is to avoid requiring communication with every tooth.

Another object of the invention is to allow both retention in the mouth as well as easy insertion and removal of the device.

Another object of the invention is to integrally form the device as a single component.

A further object of the invention is to allow movement of the lower jaw relative to the upper jaw.

Yet another object of the invention is attachment only to upper jaw teeth.

Still yet another object of the invention is simple and inexpensive fabrication.

Another object of the invention is quick and simple customization for individual patients.

Still another object of the invention is to not interfere with breathing.

Yet another object of the invention is to not interfere with speech.

Still yet another object of the invention is to be un-obvious when worn in the mouth.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, herein is disclosed a device for mitigation of temporomandibular joint disorder comprising: lingual tooth surface contact surfaces, hard palate conformity, an anterior pad, and tooth receptacles.

Numerous other objects, advantages and features of the present disclosure will be readily apparent to those of skill in the art upon a review of the following drawings and description of a preferred embodiment.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a top view of normal dentition.

FIG. 2 is a side view of the temporomandibular joint and mandible.

FIG. 3 is a perspective view of a temporomandibular joint disorder mitigation device of the present disclosure.

FIG. 4 is a top view of the temporomandibular joint disorder mitigation device of FIG. 3.

FIG. 4A is an enlarged view of tooth receptacles of the temporomandibular joint disorder mitigation device of FIG. 4 with a schematic representation of a tooth within at least one of the tooth receptacles.

FIG. 5 is a bottom view of the temporomandibular joint disorder mitigation device of FIG. 3.

FIG. 6 is a front view of the temporomandibular joint disorder mitigation device of FIG. 3.

FIG. 7 is a rear view of the temporomandibular joint disorder mitigation device of FIG. 3.

FIG. 8 is a front view of the temporomandibular joint disorder mitigation device of FIG. 3 in operative communication with human dentition.

FIG. 9 is a left perspective view of the temporomandibular joint disorder mitigation device of FIG. 3 in operative communication with human dentition.

FIG. 10 is a right perspective view of the temporomandibular joint disorder mitigation device of FIG. 3 in operative communication with human dentition.

FIG. 11 is a side cross-sectional view of human dentition showing non-appliance-adjusted occlusion.

FIG. 12 is a side cross-sectional view of a temporomandibular joint disorder mitigation device in operative communication with human dentition.

FIG. 13 is a side cross-sectional view of an alternate embodiment of temporomandibular joint disorder mitigation device in operative communication with human dentition.

FIG. 14 is a side cross-sectional view of an alternate embodiment of temporomandibular joint disorder mitigation device in operative communication with human dentition.

### LIST OF NUMBERED ELEMENTS

110 Temporomandibular joint disorder mitigation device
111 Body
112 Central incisor
114 Lateral incisor
116 Cuspid
118 Anterior teeth
120 First bicuspid
122 Second bicuspid
124 First molar
126 Second molar
128 Posterior tooth
130 Lower jaw
132 Upper jaw
134 Middle section
136 Ascending ramus
138 Coronoid process
140 Condyle
142 Meniscus
144 Articular surface of temporal bone
146 Hard palate
148 Facial tooth surface
150 Lingual tooth surface
152 Lingual tooth surface contact surface
154 Proximal tooth surface
156 Hard palate conformity
158 Lingual tooth surface conformity
160 Tooth clearance
162 Anterior pad
164 Pad wings
166 Occlusal surface
167 Incisal surface
168 U-shape or arch
170 Middle portion
172 Top surface
174 Bottom surface
176 Incline
178 Surface decline
180 Tooth receptacle
182 Facial portion
184 Lingual portion
186 Occlusal portion
188 Facial portion thickness
190 Tooth height
192 Tooth height of contour
194 Facial portion height
196 Upper end
198 Junction
199 Space
200 Pad thickness
Direction arrow A
Direction arrow B
Direction arrow C
Direction arrow D

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing. A person of skill in the art will recognize that the apparatus can assume different orientations when in use.

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal.

The instant art deals with the proper function of, and modification of or adjustment of the temporomandibular joint functions. Said functions and the anatomy of said joint, as well as pathological anomalies thereof, are well known in the art. Therefore they are not presented in stringent detail but only to the point necessary for teaching the use of the instant art.

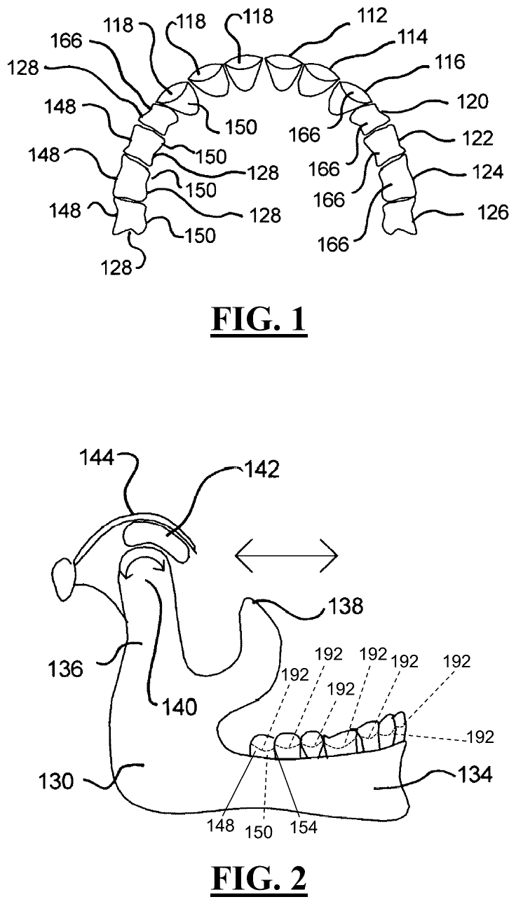

The instant art is depicted in relation to dentition of the human mouth but is not intended to be limited thereto. Also, because the instant art does not teach involvement of the third molars, also known as the wisdom teeth, such are not depicted in some drawings. Further, it is well known that particular teeth and portions thereof might have more than one name; therefore, for the purposes of this specification and these claims, the teeth and portions thereof are named as in FIG. 1 wherein the teeth are termed central incisor (112), lateral incisor (114), cuspid (116), first bicuspid (120), second bicuspid (122), first molar (124), and second molar (126). The central incisors (112), lateral incisors (114) and cuspids (116) are referred to as anterior teeth (118) and the bicuspids (120, 122) and molars (124, 126) are referred to as posterior teeth (128). Where such teeth and features are identified by reference numerals on a first side of the detention shown in FIG. 1, it will be understood that a second side (e.g., a right hand side being the first side and a left hand side being the second side) would have the same or similar reference numerals, even where such reference numerals are not shown for ease of viewing.

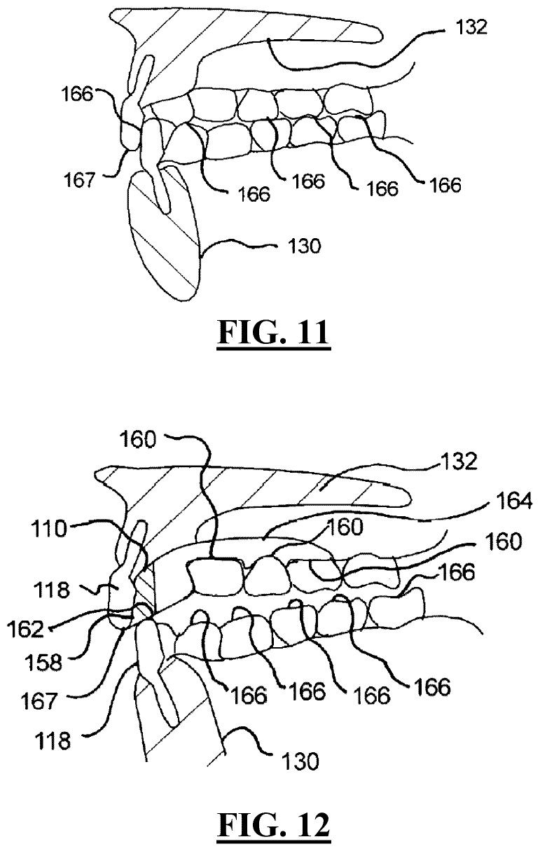

The surfaces of teeth nearest the tongue are referred to as lingual surfaces (150), the surfaces of teeth opposite the lingual surfaces (150) are referred to as facial surfaces (148), the surfaces of teeth that face adjoining teeth are referred to as proximal surfaces (154), and the surfaces of teeth which may contact or act in concert with corresponding surfaces on teeth of an opposing dental arch are referred to as occlusal surfaces (166) or incisal surfaces (167) (see FIGS. 11 and 12). Occlusal surfaces (166), as is well known in the art, comprise an occlusal plane, said occlusal plane being a well understood, familiar, easily determinable feature to one familiar with the dental arts.

As shown in FIG. 2, each tooth also has a height of contour (192), also known in the art as a height of curvature or crest of curvature. The height of contour (192) is best described as an imaginary line that circumscribes the lingual, facial, and proximal surfaces (150, 148, 154) of a tooth at the tooth's greatest bulge. The height of contour (192) is a well-known characteristic of human dentition in the relevant art. When used in reference to position or direction, the term “anterior” means toward or proximal the front of the mouth, and the term “posterior” means toward or proximal the rear of the mouth.

Those well versed in the art will readily appreciate that numbered elements of at least FIGS. 1 and 2 apply accurately to either dentition of the upper jaw or dentition of the lower jaw. The dentition, either upper or lower, intended in the specification and claims is obvious from the context.

FIG. 2 shows that the lower jaw or mandible (130) comprises a U-shaped middle section (134) which supports dentition, an ascending ramus (136) which rises substantially perpendicularly from the middle section (134) and divides into two branches, the most posterior ending in a rounded condyle (140) and the most anterior comprising a coronoid process (138) which serves as an anchor point for muscles affecting jaw movement. The condyle (140) communicates with the skull by means of a meniscus (142) interposed between said condyle (140) and an articular surface of the temporal bone (144). The condyle (140) may rotate, as indicated by curved, double-pointed arrow, thus allowing movement of the lower jaw (130) to affect opening and/or closing of the mouth. The condyle (140) may also translate, that is move forwards and/or backwards, as indicated by horizontal, double-pointed arrow.

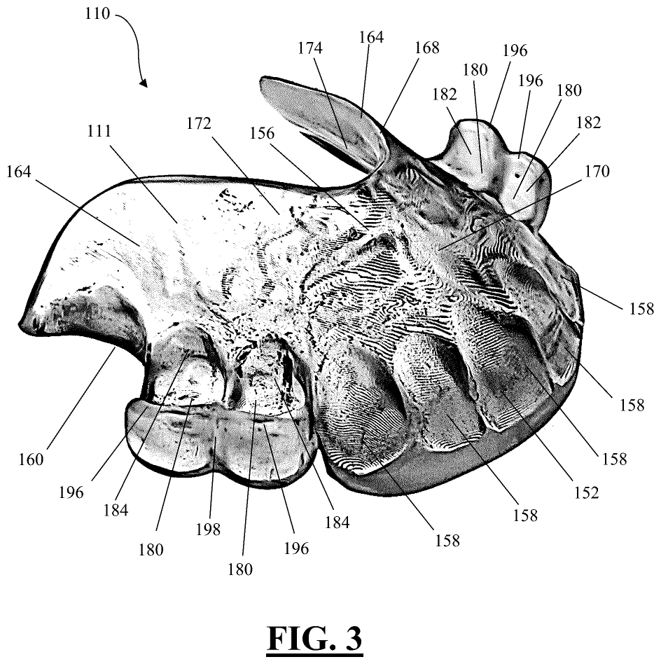

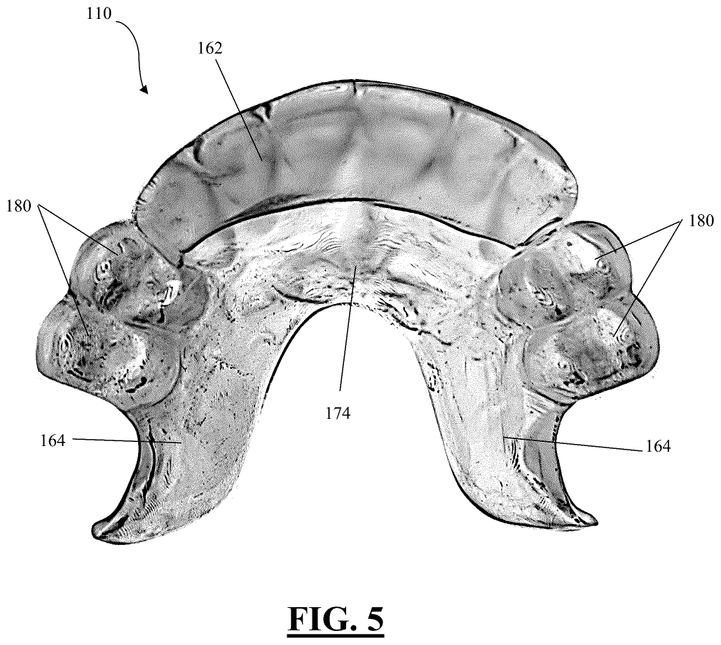

One aspect of the present disclosure is a device for mitigating temporomandibular joint disorder. FIGS. 3-7 show the temporomandibular joint disorder mitigation device (110) which includes a body (111) conformed in essentially a U-shape, or arch, (168), said U-shape or arch (168) having an anterior middle portion (170) and configured for placement in an upper jaw (132) of a subject. In some embodiments, extending posteriorly from opposite sides of the anterior middle portion (170) are pad wings (164). The whole of the device (110) is contoured to communicate with an upper jaw and dentition thereof. The device (110) comprises a top surface (172) and a bottom surface (174).

The device (110) may comprise any of known or suitable materials used in the art to fabricate sundry types of dental appliances worn either temporarily or permanently. Said material may comprise suitable flexibility, pliability, or resiliency as deemed necessary by one well versed in the art. The device (110) may comprise smooth or textured surfaces, and the device (110) may comprise material having transparency, color or pigmentation, translucence, and/or opacity. Thus, maximum comfort, minimum stress on anatomical elements interfacing with the device, and or maximum unobtrusiveness of the device (110) may be achieved.

The top surface (172) of the device (110) comprises a lingual tooth surface contact surface (152) which may further comprise one or more lingual tooth surface conformities (158), which, by means well-known in the art, may be configured to communicate essentially contiguously with the lingual surface (150), or a portion thereof, of particular teeth. In FIG. 3, six lingual tooth surface conformities (158) are noted which conform to the lingual tooth surfaces (150) of the anterior teeth (118). However, in some embodiments, the device (110) may contact the lingual tooth surface (150) of fewer or more anterior teeth (118). In some embodiments, either the lingual tooth surfaces (150) of the cuspids (116) or the lingual tooth surfaces (150) of teeth other than and/or in addition to the anterior teeth (118) may not touch the device. In other embodiments the lingual tooth surfaces (150) of the first bicuspids (120) may also communicate with the device (110).

The device (110) further comprises at least one tooth receptacle (180) extending from each side of the anterior middle portion (170). In some embodiments with pad wings (164) extending from each side of the anterior middle portion (170), the tooth receptacles (180) can extend from corresponding pad wings (164), such that they extend from the anterior middle portion (170) via the pad wings (164). In some embodiments, pad wings (164) can be thin and form an inner portion or side of the tooth receptacles (180). In other words, the thickness of the pad wings (164) may be less than the thickness of one or both of the middle portion (170) and the anterior pad (162). Each tooth receptacle (180) is shaped to receive a corresponding upper posterior tooth (128) (e.g., one of the first bicuspid (122), the first molar (124), and the second molar (126)) within the tooth receptacle (180) and has a facial portion (182) that communicates with a facial tooth surface (148) of the corresponding upper posterior tooth. In some embodiments, the tooth receptacles (180) may be integrally formed with the device (110), as described below. Each tooth receptacle (180) may engage with the corresponding upper posterior tooth (128) to retain the device (110) in an operative position in the mouth. In some embodiments, such as those depicted in FIGS. 3-10, each tooth receptacle (180) may be shaped to receive and communicate with either the first or second bicuspid (120, 122). In other embodiments, each tooth receptacle (180) may be shaped to receive a different posterior tooth (128).

The device (110) may include a hard palate conformity (156) which comprises the top surface (172) of the pad wings (164) where applicable and that portion of the top surface (172) of the device middle portion (170) posterior to the lingual tooth surface conformity (158). Thus, it may be readily appreciated that the hard palate conformity (156) comprises an unbroken arch (168). The device (110) further includes at least one tooth clearances (160) located within the pad wings (164) for one or more posterior teeth. FIGS. 3-7 depict pad wings (164) that each include one tooth clearance (160), but the device (110) may include tooth clearances (160) for more posterior teeth (128) to conform to the mouth of a given wearer. The tooth clearances (160) render the device (110) as simple, small, unobtrusive, and comfortable as possible.

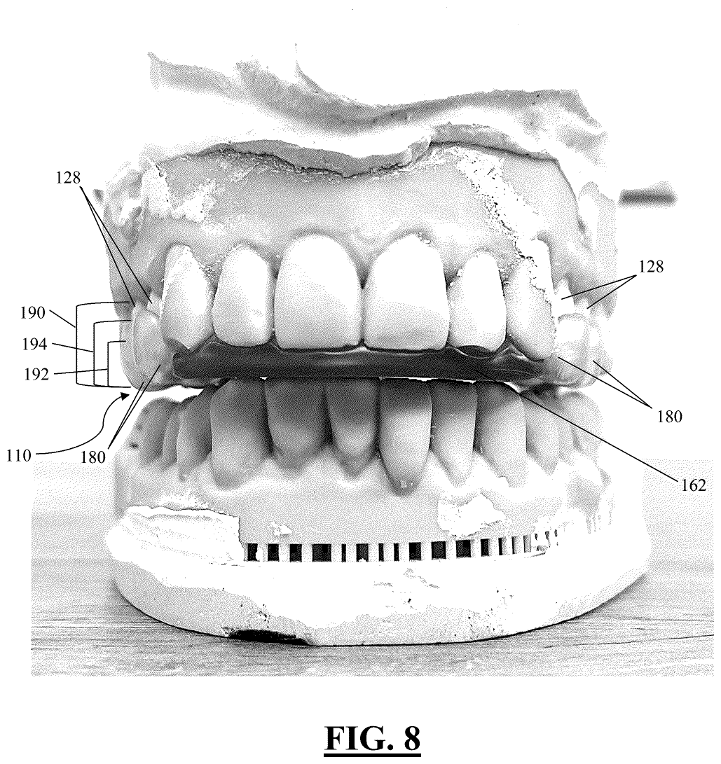

In some embodiments, as shown in FIGS. 3-10, each tooth receptacle (180) may further include a lingual portion (184) and an occlusal portion (186) that communicate with the lingual surface (150) and the occlusal surface (166) respectively of the corresponding upper posterior tooth (128) (see FIGS. 8-10). In other embodiments, each tooth receptacle (180) may include no occlusal portion (186) and/or no lingual portion (184). For example, each tooth receptacle (180) may be connected to the pad wings (164) via one or more proximal portions (187) that conform to either the spaces between the occlusal surfaces (166) or the proximal surfaces (154) of the corresponding upper posterior teeth.

Using tooth receptacles (180) to retain the device (110) in an operative position in the mouth can greatly reduce or eliminate any rocking or pivoting movement of the device (110) that may otherwise occur, particularly when ball clasps are used to secure the device (110) in the mouth, and can also prevent any slippage that may occur when friction is the sole force retaining the device (110) in an operative position. Thus, the device (110) may be retained in the optimal position for mitigating temporomandibular joint disorder and the wearer need not continuously reposition the device (110). Further, flexural stresses on the device (110) during insertion into and removal from the mouth may be distributed to all the material that forms the tooth receptacles (180), especially in comparison to ball clasps and other retention members that are not integrally formed with the device (110) and/or that are formed from thin metal members. Accordingly, the tooth receptacles (180) are unlikely to weaken and lose retention power over time due to repeated flexing during insertion and removal of the device (110).

As noted above, in some embodiments, the tooth receptacles (180) may be integrally formed with the device (110), meaning the entirety of the device (110), including all elements described herein, may be fabricated as a single component and not two or more components attached or assembled together. This allows the device (110) to be formed more quickly and easily than similar devices that require multiple components to be separately formed and/or assembled. For example, the device (110) may be formed by a milling or machining process, whereby a single piece of material is shaped by cutting away material to form the device (110). The device (110) may also be formed by a molding process, whereby a mold is created of the desired shape of the device (110) and the material that forms the device (110) adopts the shape of the mold. The material, in liquid or otherwise pliable form, may be either inserted into a hollow mold or heated over a mold, then the material may harden or set in the shape of the device (110). The most efficient process for manufacturing the device (110) may be 3D printing or additive manufacturing, whereby, generally, a three-dimensional computer model of the device (110) is created and a machine deposits, joins, or solidifies the material in the shape of the three-dimensional computer model of the device (110). The list of manufacturing processes briefly described herein is not exhaustive, and any suitable process may be employed to integrally form the device as a single component.

While the device (110) as shown in FIGS. 3-10 includes two tooth receptacles (180) extending from each of the pad wings (164), in some embodiments, the device (110) may include more or fewer tooth receptacles (180) extending from each of the pad wings (164). The embodiment depicted in FIGS. 3-10 also shows tooth receptacles (180) positioned adjacent to each other, but in some embodiments that include a plurality of tooth receptacles (180), the tooth receptacles (180) may be spaced apart from each other. Some wearers of the device (110) may be missing one or more upper posterior teeth (128) on one or both sides of the upper jaw (132), and other wearers may have upper posterior teeth (128) shaped such that additional retention support is necessary. The device (110) may thus include the appropriate number of tooth receptacles (180) positioned to engage with corresponding suitably-shaped teeth such that the device (110) may be adequately retained in an operative position for each individual wearer.

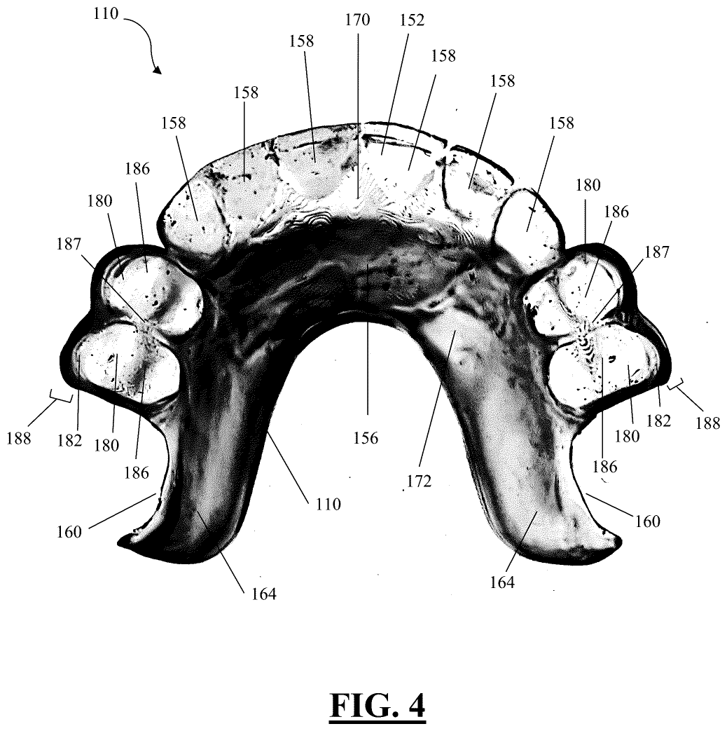

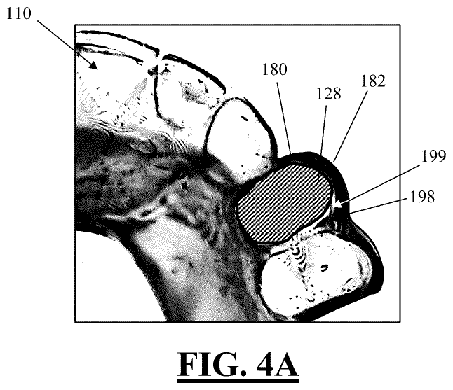

In some embodiments, as best shown in FIG. 4, the facial portion (182) of each of the tooth receptacles (180) may have a facial portion width (188) such that the device (110) has a rigidity that allows for retention in the mouth and a flexibility that allows for easy insertion into and removal from the mouth. The optimal facial portion width (188) is between 1.5 millimeters and 2 millimeters. The width (188) may in other words be a thickness of the facial portion (182). Each tooth receptacle (180) may be shaped, sized, or otherwise configured to retain the respective tooth within the tooth receptacle (180) without placing forces on the corresponding tooth that may cause the tooth to move. For example, the internal surfaces, such as the surface of the facial portion (182) of the tooth receptacles (180) may be shaped, sized, or otherwise configured to retain the corresponding tooth against the force of gravity (180) without moving the corresponding tooth. In some embodiments, the force applied by the tooth receptacle(s) is sufficient to retain the corresponding tooth without the tooth receptacle (180) moving the corresponding tooth or other teeth within the mouth, notwithstanding the movement caused by the other portions of the device (110). In some embodiments, the force may be less than or equal to about 0.10 Newtons (N), less than or equal to about 0.15 N, less than or equal to about 0.20 N, less than or equal to about 0.25 N, less than or equal to about 0.30 N, less than or equal to about 0.35 N, less than or equal to about 0.40 N, less than or equal to about 0.45 N, less than or equal to about 0.5 N, less than or equal to about 0.10 N to about 0.5 N, or any value or range of values therebetween that is selected to prevent movement of the tooth being received by the tooth receptacle (180).

In some embodiments wherein each tooth receptacle (180) includes an occlusal portion (186), the occlusal portion (186) of each of the tooth receptacles (180) may have an occlusal portion width such that the tooth receptacle (180) does not contact the lower posterior teeth when the device (110) is in an operative position in the mouth. In some embodiments, the device (110) may be formed such that the tooth receptacle(s) (180) is spaced apart from the lower posterior teeth when the device (110) is in an operative position in the mouth. It will be understood that the space between the tooth receptacle(s) may be any size, including being as small as to not be visible on a visual inspection of the device (110). In some embodiments, the device (110) may be formed such that the lower posterior teeth do not substantially engage the tooth receptacle(s) (128) when the device (110) is in an operative position in the mouth.

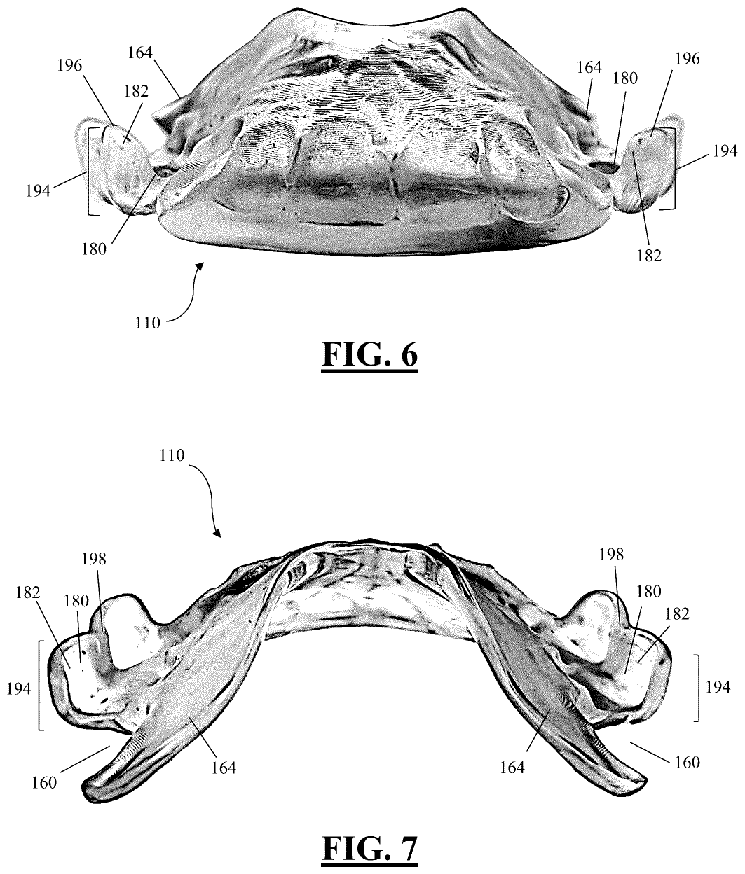

As best shown in FIGS. 6 and 7, the facial portion (182) of each of the tooth receptacles (180) also has a facial portion height (194) and an upper end (196). Each tooth has a height of contour (192), as described above, and, as shown using one of the tooth receptacles (180) and the respective tooth (128) as an example, a tooth height (190). In some embodiments, the facial portion height (194) of each tooth receptacle (180) may be greater than the height of contour (192) but less than the tooth height (190) of the corresponding tooth. While the heights (190, 192, 194) are shown only for one of the tooth receptacles (180) in FIG. 8, it will be understood that similar measurements may be taken for each tooth receptacle (180) and the corresponding tooth (128).

For each tooth receptacle (180) receiving an upper posterior tooth (128), the facial portion height (194) being greater than the tooth's height of contour (192) allows the device (110) to be retained in its operative position because the tooth receptacle (180) cannot flex to the extent necessary to clear the height of contour (192) without some application of force. The facial portion height (194) being less than the tooth height (190) allows for the device (110) to be removed from the wearer's mouth by applying force to the upper end (196) of the facial portion (182) without the device or the source of the force contacting the gumline, both of which can cause irritation and bleeding. Most often, the wearer will use his or her finger or fingernail to apply force to the upper end (196) of the facial portion (182) of one of the tooth receptacles (180) to remove the device (110) from its operative position in the mouth. The optimal difference between the facial portion height (194) of each of the tooth receptacles (180) and the height of contour (192) of the corresponding tooth is between 2 millimeters and 3 millimeters.

Referring again to FIGS. 3-7, in some embodiments wherein the device (110) includes at least two tooth receptacles (180) extending from at least one pad wing (164) and positioned adjacent to each other, the device may further include a junction (198) located between each of the facial portions (182) of the tooth receptacles (180). At least part of the junction (198) may be spaced from the facial surfaces (148) of the corresponding teeth by a distance or space (199) (see FIG. 4A). The optimal measurement of the distance or space (199) by which each junction (198) is spaced from the facial surface (148) of the corresponding tooth (129) is about 50 to about 100 micrometers. The distance allows for the tooth receptacles (180) to contact a sufficient amount of the facial surfaces (148) of the corresponding teeth to ensure retention while also allowing for easy insertion and removal of the device (110). In some embodiments, the junction (198) may have a junction height that is less than the heights of the proximal surfaces (154) but greater than the heights of contour (192) of both of the corresponding teeth such that the device (110) is not excessively retained. Additionally, in some embodiments, the junction (198) may have a junction width sufficient to endure flexural stresses when force is applied to the upper end (196) of the facial portion (188) of one of the adjoining tooth receptacles (180).

Referring now to FIGS. 8-14, in operative position, the device (110) communicates substantially contiguously with the lingual and occlusal surfaces of the anterior teeth (118) and the hard palate (146). Additionally, it may be noted that the posterior teeth (128), particularly the first bicuspid (120), second bicuspid (122), and first molar (124), do not communicate with the pad wings (164) (see FIGS. 3-7), due to the presence of, in some embodiments, the tooth receptacles (180), or, in other embodiments, the tooth clearances (160) (see FIGS. 3-7).

As shown in FIGS. 3-7, the device (110) further includes an anterior pad (162) comprising the bottom surface (174) of the middle portion (170) of the arch (168) of the device (110). Also as shown in FIGS. 8-14, the device (110) can communicate with the occlusal surfaces (166) of the posterior teeth (128), or the incisal surfaces (167) of the anterior teeth (118), but, as shown in FIG. 12, need not so communicate with such surfaces.

In addition, it may be noted that function of the device (110) does not require it to be in communication with the roof of the mouth or soft palate. In other words, in various examples, the device (110), when disposed in the operative position, may be positioned adjacent to the roof of the mouth or the soft palate without overlapping the soft palate.

FIG. 11 shows that the upper jaw (132) and the lower jaw or mandible (130) may contiguously interface at occlusal surfaces (166) naturally. Thus, occasion may be created for temporomandibular joint disorder causing undesirable events or conditions, in example bruxism or forward thrusting of the lower jaw (130).

FIGS. 8-10 and 12 show that when the device (110) is disposed in operative position, the anterior pad (162) is interposed between the upper jaw (132) anterior teeth (118) and the lower jaw (130) anterior teeth (118) to serve the function of limiting the range of upward vertical extension of the lower jaw (130). The range of upward vertical extension of the lower jaw (130) is limited such that there is neither occlusion of occlusal surfaces (166) of posterior teeth (128), nor contact between the lower posterior teeth and any part of the device (110), including the tooth receptacles (180). Also noted is that the anterior teeth (118) are free to contact and move about on the surface of the anterior pad (162).

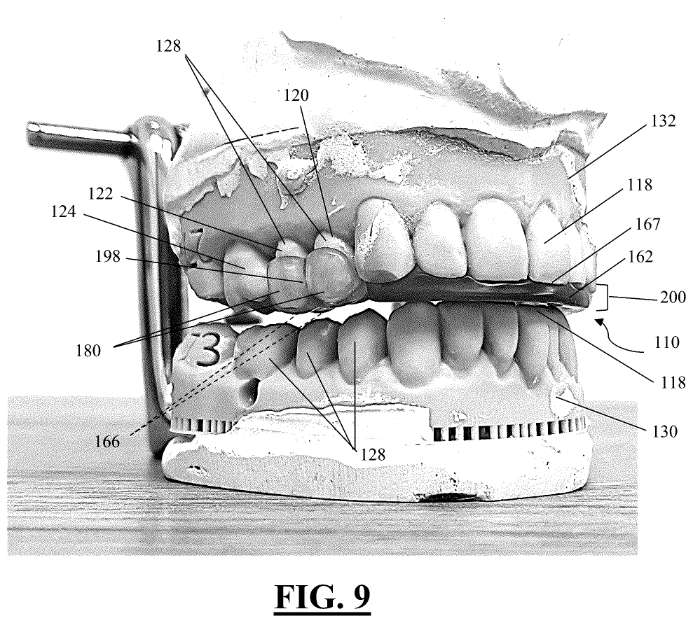

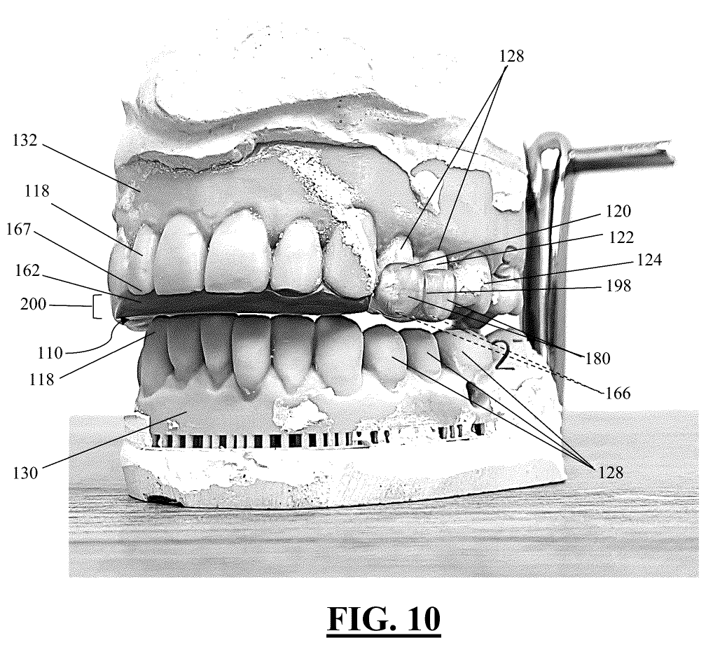

In addition, the flexibility, pliability, and/or resiliency of the material comprising the device may serve to cushion the contact between the anterior pad (162) and the lower jaw anterior teeth (118). Noted also in FIG. 12 is that the anterior pad is disposed essentially parallel to the occlusal plane of the anterior teeth (118). The anterior pad (162) may be configured to have a thickness (200) such that only the anterior teeth (118) of the lower jaw (130) contact the anterior pad (162) and the posterior teeth (128) of the lower jaw (130) do not contact the tooth receptacles (180) (see FIGS. 9 and 10).

In this manner, bruxism and its attendant damage causing stress on the temporomandibular joint are mitigated. This also allows opportunity for any previous temporomandibular joint damage is given to heal. Further, the previously mentioned temporomandibular joint disorder associated symptoms, especially the pains in various localities, may be significantly alleviated if not altogether eliminated.

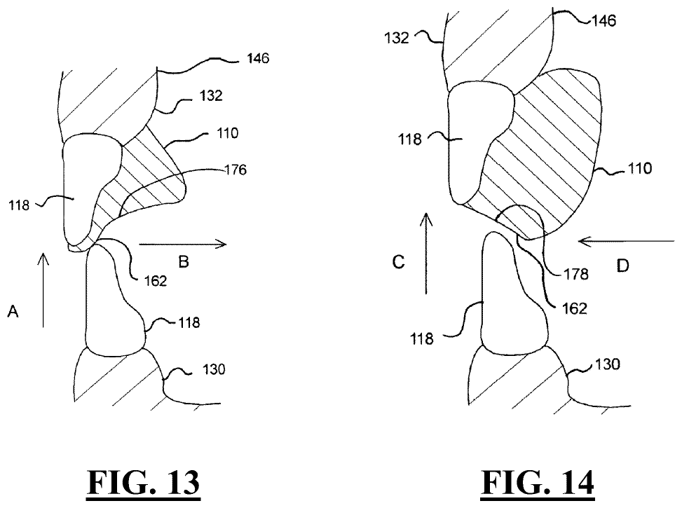

Referring to FIG. 13, one may readily appreciate that movement of the anterior teeth (118) relative the anterior pad (162) may be freely accomplished by translational movement of the lower jaw (130).

FIG. 13 shows that the anterior pad (162) may be configured so as to comprise an incline (176) from anterior to posterior. In other words, the anterior pad (162) may comprise a contour that inclines from anterior to posterior. So configured, when a lower anterior tooth (118) is thrust against the anterior pad (162) in direction A, as indicated by arrow A, a force B, as indicated by arrow B, essentially toward the posterior, will result. Thus, as the lower jaw (130) closes, it is naturally forced toward the posterior.

In this manner, thrusting of the lower jaw (130) and its attendant damage-causing stress on the temporomandibular joint are mitigated. Thus, any previous temporomandibular joint damage is given opportunity to heal, and the previously mentioned temporomandibular joint disorder associated symptoms, especially the pains in various localities, may be significantly alleviated if not altogether eliminated.

Although the device (110) is depicted as having an anterior pad (162) extending essentially horizontally or at an incline from anterior to posterior, the instant art is not intended to be thusly limited. In particular, an anterior pad (162), as in FIG. 14, may comprise a surface declining (178) from anterior to posterior, thusly creating a force in direction C, toward the anterior, as indicated by arrow C, when force in direction D, as indicated by arrow D, causes lower jaw (130) anterior teeth (118) to encounter the anterior pad (162). In other words, the anterior pad (162) may comprise a contour that declines from anterior to posterior. Such disposition could be utilized for a patient with an under bite.

So configured, one may readily appreciate that movement of the anterior teeth (118) relative the anterior pad (162) may be freely accomplished by voluntary forward translational movement of the lower jaw (130).

Another aspect of the present disclosure is a method for forming a single-component device (110) for mitigation of temporomandibular joint disorder. In other words, the method described herein provides for the manufacture of a device (110) wherein all elements of the device are integrally formed. The method comprises the first step of taking an impression of the upper jaw teeth and hard palate. This step must be done for each individual patient, and any suitable process for taking an impression of the required mouth portions will suffice. The method further comprises the second step of creating a three-dimensional computer upper jaw model of the impression of the upper jaw teeth and hard palate. A number of computer programs exist to create digital models of dental impressions, and any suitable program may be employed.

The method further comprises the third step of creating a three-dimensional computer device model configured to conform with the three-dimensional computer upper jaw model. The three-dimensional computer device model includes a middle portion, pad wings extending posteriorly from the middle portion, and at least one tooth receptacle extending from each of the pad wings. However, the three-dimensional computer device model does not include any protrusive elements extending from the bottom surface of the middle portion and that are configured to engage lingual surfaces of the anterior lower jaw teeth. An example of such a device is described in detail above.

Finally, the method further comprises the fourth step of manufacturing the device (110) to conform to the three-dimensional computer device model such that the device, including all elements thereof, is integrally formed as a single component. Some benefits of manufacturing such a device to be integrally formed are described in detail above, but the key benefit with respect to manufacturing the device in particular is that all elements of the device may be formed at one point in time, as no additional components must be attached or assembled together at any point during the process. Manufacturing methods employed at this step may include milling or machining, injection, compression, melt, or other types of molding, 3D printing or additive manufacturing, or any other method suitable for integrally forming the device.

The optimal manufacturing method to accomplish the fourth step is 3D printing. The 3D printer to be used need only be large enough to produce the device, which is relatively small and fits in a human mouth. Accordingly, it could easily be located in the same location where all other steps of the method are performed, such as a dentist office. When this is the case, no additional time must be spent transmitting the three-dimensional computer device model to a manufacturing location and subsequently transporting the device from the manufacturing location to its final destination, which is often either the dentist office where the impression was taken or the patient's home. All steps of the method may thus be performed potentially in one day or in a few days, such that a patient may seek fast relief from temporomandibular joint disorder.

Even when the 3D printer is not located in the same place where all the other steps are performed, 3D printing requires minimal human effort to produce the device. The three-dimensional computer device model may be digitally transmitted to the 3D printer and thereafter, the device may be 3D printed to precisely conform to the three dimensional computer device model in a single process. Additionally, 3D printers may print a variety of materials suitable for forming all elements of the device.

While the inventions are described herein in connection with preferred embodiments, this description is not intended to limit the scope of the invention to the particular form set forth, but on the contrary, it is intended to cover such alternatives, modifications, and equivalents as may be included within the spirit and scope of the invention as defined by the appended claims.

Thus, although there have been described particular embodiments of the present invention of a new and useful device for mitigation of temporomandibular joint disorder, it is not intended that such references be construed as limitations upon the scope of this invention.

What is claimed is:

1. A device for mitigation of temporomandibular joint disorder comprising:
a middle portion having a top surface and a bottom surface, the top surface being configured to communicate with lingual surfaces of one or more of a user's anterior upper jaw teeth and hard palate, the bottom surface of the middle portion being comprised of an anterior pad that is configured such that movement of one or more anterior lower jaw teeth of the user relative to the anterior pad can be freely accomplished by translational movement of the user's lower jaw; and
at least one tooth receptacle extending the middle portion, each tooth receptacle shaped to receive a corresponding upper posterior tooth within the tooth receptacle and having a facial portion that communicates with a facial surface of the corresponding upper posterior tooth;
wherein the anterior pad has a thickness such that the anterior lower jaw teeth contact the anterior pad and the posterior lower teeth do not substantially engage the tooth receptacles.

2. The device of claim 1, wherein the anterior pad comprises a contour that inclines from anterior to posterior.

3. The device of claim 1, wherein the anterior pad comprises a contour that declines from anterior to posterior.

4. The device of claim 1, wherein said top surface is configured to communicate with at least one occlusal surface of at least one anterior upper jaw tooth.

5. The device of claim 1, wherein a portion of the device that engages the anterior teeth of the user's upper jaw is configured to only engage the lingual surfaces of the anterior teeth of the user's upper jaw.

6. The device of claim 1, wherein the anterior pad is configured such that movement of the anterior lower jaw teeth relative to the anterior pad can be freely accomplished by voluntary forward translational movement of the lower jaw.

7. The device of claim 1, wherein the device is conformed substantially in a U-shape, and wherein the device is positioned adjacent a soft palate of the user.

8. The device of claim 1, wherein each tooth receptacle is integrally formed with the middle portion.

9. The device of claim 8, wherein the tooth receptacles are configured to retain the device in an operative position in the user's mouth.

10. The device of claim 1, wherein each tooth receptacle further includes a lingual portion and an occlusal portion that communicate with a lingual surface and an occlusal surface respectively of the corresponding upper posterior tooth.

11. The device of claim 1, wherein the facial portion of the at least one tooth receptacle has a facial portion width such that the device has a rigidity that allows for retention in the user's mouth and a flexibility that allows for easy insertion into and removal from the user's mouth.

12. The device of claim 11, wherein the facial portion width of the at least one tooth receptacle is about 1.5 millimeters to about 2 millimeters, and wherein the at least one tooth receptacle is configured to apply a force to retain the corresponding tooth within the at least one tooth receptacle without moving the corresponding tooth or other teeth of the user.

13. The device of claim 1, wherein:
each upper posterior tooth has a tooth height and a height of contour;
the facial portion of the at least one tooth receptacle has a facial portion height and an upper end; and
the facial portion height is greater than the height of contour but less than the tooth height of the corresponding tooth such that the device may removed from the user's mouth by applying force to the upper end of the facial portion without the source of the force contacting the user's gumline.

14. The device of claim 13, wherein the difference between the facial portion height of the at least one tooth receptacle and the height of contour of the corresponding tooth is about 2 millimeters to about 3 millimeters.

15. The device of claim 1, wherein the at least one tooth receptacle comprises at least two tooth receptacles extending from at least one pad wing and positioned adjacent to each other, and a junction is located between each of the facial portions of the tooth receptacles, wherein at least a portion of each junction is spaced from the facial surfaces of the corresponding teeth by a distance.

16. The device of claim 1, further comprising pad wings extending posteriorly from corresponding opposite extremes of the middle portion, said pad wings having a top surface configured to communicate with the hard palate of the user's upper jaw structure and tooth clearances for one or more posterior teeth, wherein each of the at least one tooth receptacles extends from a corresponding pad wing.

17. A device, comprising:
a main U-shaped body configured for placement in an upper jaw of a subject that includes a middle portion, and at least one tooth receptacle extending from the middle portion and shaped to receive a corresponding upper posterior tooth;
a top surface of the middle portion configured to communicate with a hard palate of the subject and lingual surfaces of four or more anterior teeth of the upper jaw; and
a bottom surface of the middle portion having an anterior pad, the anterior pad configured such that voluntary forward translational movement of the lower jaw teeth relative to the body can be freely accomplished.

18. The device of claim 17, wherein the body is positioned adjacent to at least one of a soft palate of the subject and the posterior teeth of the subject's lower jaw.

19. The device of claim 17, wherein a portion of the device that communicates with the anterior teeth of the upper jaw engages the lingual surfaces of the anterior upper jaw teeth.

20. The device of claim 17, further comprising:
one or more pad wings extending from the middle portion and having a top surface configured to communicate with the hard palate of the subject and tooth clearances for one or more posterior teeth,
wherein the at least one tooth receptacle extends from the one or more pad wings.
